(19) United States
(12) Patent Application Publication
(10) Pub. No.: US 2023/0337980 A1
(43) Pub. Date: Oct. 26, 2023

(54) PORTABLE RAPID DETECTION DEVICE FOR COGNITION, SLEEP AND PSYCHOLOGICAL STATUS

(71) Applicants: Air Force Medical University, Xi'an City (CN); Shaanxi Shenger Yaxin Electronic Techn. Co., Ltd., Xi'an City (CN)

(72) Inventors: Wen WANG, Xi'an City (CN); Guangbin CUI, Xi'an City (CN); Linfeng YAN, Xi'an City (CN); Ying YU, Xi'an City (CN); Bo HU, Xi'an City (CN); Zhuhong CHEN, Xi'an City (CN); Jingting SUN, Xi'an City (CN); Zeshun WU, Xi'an City (CN)

(21) Appl. No.: 18/301,410

(22) Filed: Apr. 17, 2023

(30) Foreign Application Priority Data
Apr. 19, 2022 (CN) 202210407417.4

(51) Int. Cl. A61B 5/00; A44C 5/00

(52) U.S. Cl. CPC A61B 5/681; A61B 5/0059; A44C 5/0023

(57) ABSTRACT

Disclosed is a portable rapid detection device for cognition, sleep and psychological status. The portable rapid detection device comprises a detection dial plate, an adjusting knob, woven watchbands, screw rods, sweat-absorbent linings and a sweat-absorbent quick-drying pad. The detection dial plate is of a cylindrical structure. The embedded adjusting knob is installed in the middle of the front end of the detection dial plate. The woven watchbands are installed at two ends of the detection dial plate and fixed through the screw rods. The embedded sweat-absorbent lining is installed on the inner side of the woven watchband. The sweat-absorbent quick-drying pad is installed in the middle of the bottom of the detection dial plate and fixed through magnetic attraction.

### TECHNICAL FIELD

The present disclosure relates to a portable rapid detection device, in particular to a portable rapid detection device for cognition, sleep and psychological status.

### BACKGROUND

With the acceleration of the pace of social life, modern people pay more and more attention to personal health. At the same time, detection devices for night sleep, motion status and psychological status are constantly updated. At present, most of the detection devices wear on wrists in summer and sweat is caused, so that a wearing experience of a user is influenced. Therefore, a novel rapid detection device for cognition, sleep and psychology is needed to improve the experience of the user when sweating.

### SUMMARY

The present disclosure aims to provide a portable rapid detection device for cognition, sleep and psychological status to solve the above technical problems.

To achieve the above purpose, the present disclosure adopts the following technical scheme.

A portable rapid detection device for cognition, sleep and psychological status includes a detection dial plate, an adjusting knob, woven watchbands, screw rods, sweat-absorbent linings and a sweat-absorbent quick-drying pad. The detection dial plate is of a cylindrical structure. The embedded adjusting knob is installed in the middle of the front end of the detection dial plate. The woven watchbands are installed at two ends of the detection dial plate and fixed through the screw rods. The embedded sweat-absorbent lining is installed on the inner side of the woven watchband. The sweat-absorbent quick-drying pad is installed in the middle of the bottom of the detection dial plate and fixed through magnetic attraction.

Based on the technical scheme, the woven watchband includes a woven fabric outer layer, a bactericidal layer, a waterproof layer and adjusting elastic bands. An inner side of the woven fabric outer layer is filled with the bactericidal layer of a rectangular cavity structure. An inner side of the bactericidal layer is filled with the waterproof layer. The adjusting elastic bands equidistantly and symmetrically distributed are installed on the inner side of the waterproof layer.

Based on the technical scheme, the sweat-absorbent quick-drying pad includes a magnetic sticker, a rubber pad, a heating sheet, telescopic rods, a rubber ring and a sweat-absorbent cotton pad. A bottom cover plate of the detection dial plate is made of metal. An embedded light sensing detection device is installed in the middle of the bottom cover plate of the detection dial plate. An embedded charging port is installed on the outer side of the detection device. The magnetic sticker of a cylindrical structure is installed on the top of the charging port. The rubber pad is installed on the top of the magnetic sticker. The heating sheet is installed in the middle of the top of the magnetic sticker. The telescopic rods equidistantly and symmetrically distributed are installed on the outer side of the heating sheet. The rubber ring is installed on the top of the telescopic rod and fixed by bonding. An inner side of the rubber ring is bonded with the sweat-absorbent cotton pad of a boss structure.

Compared with the prior art, the present disclosure has the following advantages. According to the detection device in the present disclosure, the woven watchband connected through the screw rod is convenient to replace, and sweat can be quickly absorbed through a woven fabric and the sweat-absorbent lining. At the same time, the waterproof layer and the adjusting elastic bands can prevent sweat from corroding the elastic bands and prolong the service life while being adapted to the wrist. Then, the sweat-absorbent quick-drying pad can avoid the phenomenon that the sweat at the bottom of the detection dial plate is not easy to volatilize. Meanwhile, the rubber pad, the telescopic rods and the rubber ring can form circulation cooling gaps to accelerate heat dissipation. When the dial plate is taken off, power can be supplied for the heating sheet through the dial plate to dry and sterilize the sweat-absorbent cotton pad, so that the harm caused by bacterial growth to the body of a user is further avoided.

### BRIEF DESCRIPTION OF THE DRAWINGS

FIG. 1 is a structural schematic diagram of an appearance of the present disclosure.

FIG. 2 is a connecting structural schematic diagram of woven watchbands in the present disclosure.

FIG. 3 is an enlarged structural schematic diagram of the woven watchband in the present disclosure.

FIG. 4 is a structural schematic diagram of a sweat-absorbent quick-drying pad in the present disclosure.

Reference signs: 1, detection dial plate; 1-1, detection device; 1-2, charging port; 2, adjusting knob; 3, woven watchband; 4, screw rod; 5, sweat-absorbent lining; 6, sweat-absorbent quick-drying pad; 7, woven fabric outer layer; 8, bactericidal layer; 9, waterproof layer; 10, adjusting elastic band; 11, magnetic sticker; 12, rubber pad; 13, heating sheet; 14, telescopic rod; 15, rubber ring; and 16, sweat-absorbent cotton pad.

### DETAILED DESCRIPTION OF THE EMBODIMENTS

The present disclosure is further described in detail in conjunction with the attached figures and specific embodiments.

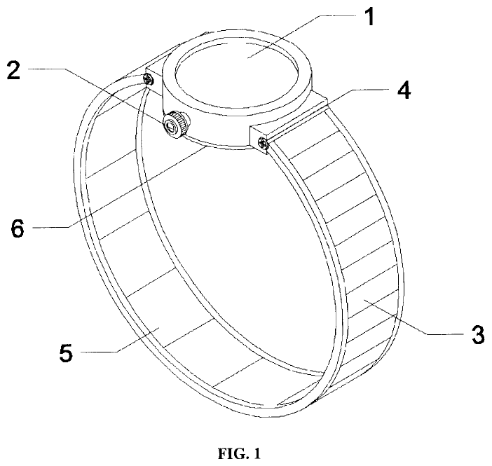

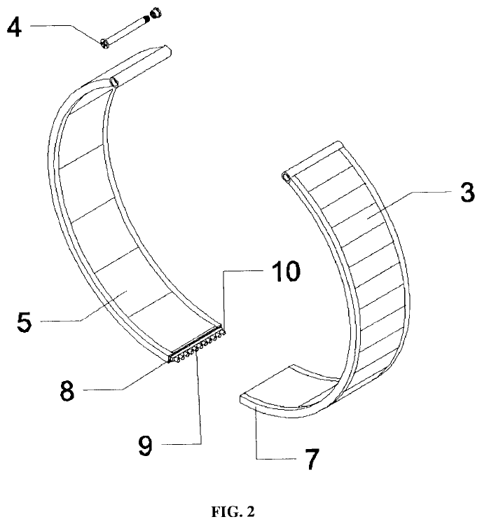

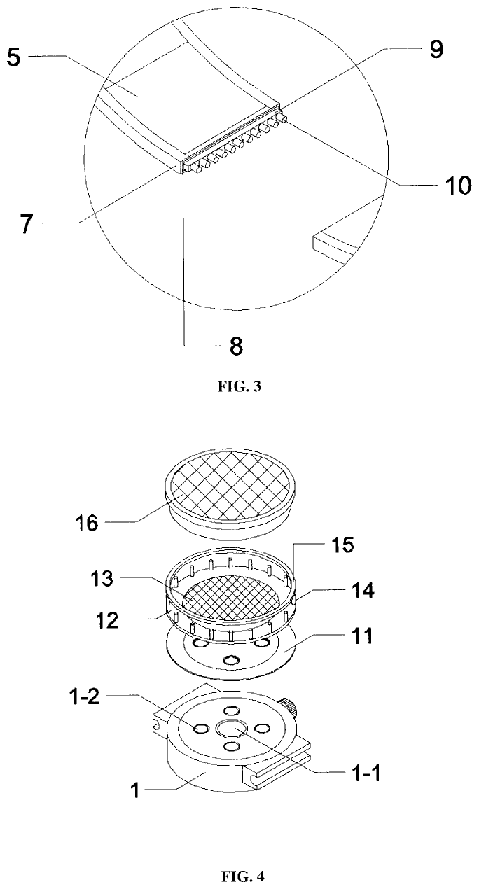

As shown in FIG. 1 to FIG. 4, a portable rapid detection device for cognition, sleep and psychological status includes a detection dial plate 1, an adjusting knob 2, woven watchbands 3, screw rods 4, sweat-absorbent linings 5 and a sweat-absorbent quick-drying pad 6. The detection dial plate 1 is of a cylindrical structure. The embedded adjusting knob 2 is installed in the middle of a front end of the detection dial plate 1. The woven watchbands 3 are installed at two ends of the detection dial plate 1 and fixed through the screw rods 4. The embedded sweat-absorbent lining 5 is installed on the inner side of the woven watchband 3. The sweat-absorbent quick-drying pad 6 is installed in the middle of the bottom of the detection dial plate 1 and fixed through magnetic attraction.

The woven watchband 3 includes a woven fabric outer layer 7, a bactericidal layer 8, a waterproof layer 9 and adjusting elastic bands 10. An inner side of the woven fabric outer layer 7 is filled with the bactericidal layer 8 of a rectangular cavity structure. An inner side of the bactericidal layer 8 is filled with the waterproof layer 9. The adjusting elastic bands 10 equidistantly and symmetrically distributed are installed on the inner side of the waterproof layer 9.

The sweat-absorbent quick-drying pad 6 includes a magnetic sticker 11, a rubber pad 12, a heating sheet 13, telescopic rods 14, a rubber ring 15 and a sweat-absorbent cotton pad 16. A bottom cover plate of the detection dial plate 1 is made of metal. An embedded light sensing detection device 1-1 is installed in the middle of the bottom cover plate of the detection dial plate 1. An embedded charging port 1-2 is installed on the outer side the detection device 1-1. The magnetic sticker 11 of a cylindrical structure is installed on the top of the charging port 1-2. The rubber pad 12 is installed on the top of the magnetic sticker 11. The heating sheet 13 is installed in the middle of the top of the magnetic sticker 11. The telescopic rods 14 equidistantly and symmetrically distributed are installed on the outer side of the heating sheet 13. The rubber ring 15 is installed on the top of the telescopic rod 14 and fixed by bonding. An inner side of the rubber ring 15 is bonded with the sweat-absorbent cotton pad 16 of a boss structure.

The working principle of the present disclosure is as follows. The watchbands are inserted into grooves in two ends of the dial plate and then locked by the screw rods, and then the elastic bands inside the woven watchbands can be adjusted in real time according to the shape of a wrist, so that the wrist is more comfortable and natural to be fitted. Then, when the wrist sweats in summer, sweat can be absorbed and transferred to the inner side of a woven fabric through the sweat-absorbent lining, and at the same time, the woven fabric is sterilized by the waterproof layer and the bactericidal layer to prevent the sweat from corroding the elastic bands and prolong the service life of the watchband. Then, the magnetic sticker is connected with the cover plate of the detection dial plate made of metal. Meanwhile, the rubber pad, the telescopic rods and the rubber ring can form circulation cooling holes for the sweat-absorbent cotton pad to accelerate heat dissipation of the cotton pad. When the detection device is taken off, power can be supplied for the heating sheet through the dial plate, and then the heating sheet can sterilize and dry the sweat-absorbent cotton pad to improve the safety factor.

Although the embodiments of the present disclosure have already been illustrated and described, various changes, modifications, replacements and transformations can be made by those skilled in the art under the condition of without departing from the principle and the spirit of the present disclosure, and thus the scope of the present disclosure should be restricted by claims and equivalents thereof.

1. A portable rapid detection device for cognition, sleep and psychological status, comprising:
a detection dial plate,
an adjusting knob,
woven watchbands,
screw rods,
sweat-absorbent linings, and
a sweat-absorbent quick-drying pad,
wherein the detection dial plate is of a cylindrical structure, the embedded adjusting knob is installed in the middle of a front end of the detection dial plate, the woven watchbands are installed at two ends of the detection dial plate and fixed through the screw rods, the embedded sweat-absorbent lining is installed on the inner side of the woven watchband, and the sweat-absorbent quick-drying pad is installed in the middle of the bottom of the detection dial plate and fixed through magnetic attraction.

2. The portable rapid detection device according to claim 1, wherein the woven watchband comprises a woven fabric outer layer, a bactericidal layer, a waterproof layer and adjusting elastic bands, an inner side of the woven fabric outer layer is filled with the bactericidal layer of a rectangular cavity structure, and an inner side of the bactericidal layer is filled with the waterproof layer, and the adjusting elastic bands equidistantly and symmetrically distributed are installed on the inner side of the waterproof layer.

3. The portable rapid detection device according to claim 1, wherein the sweat-absorbent quick-drying pad comprises a magnetic sticker, a rubber pad, a heating sheet, telescopic rods, a rubber ring and a sweat-absorbent cotton pad, a bottom cover plate of the detection dial plate is made of metal, an embedded light sensing detection device is installed in the middle of the bottom cover plate of the detection dial plate, an embedded charging port is installed on the outer side the detection device, the magnetic sticker of a cylindrical structure is installed on the top of the charging port, the rubber pad is installed on the top of the magnetic sticker, the heating sheet is installed in the middle of the top of the magnetic sticker, the telescopic rods equidistantly and symmetrically distributed are installed on the outer side of the heating sheet, the rubber ring is installed on the top of the telescopic rod and fixed by bonding, and an inner side of the rubber ring is bonded with the sweat-absorbent cotton pad of a boss structure.
